(19) United States
(12) Patent Application Publication
(10) Pub. No.: US 2025/0102433 A1
(43) Pub. Date: Mar. 27, 2025

(54) FIBER OPTIC BIOSENSOR FOR ULTRA-LOW TRACE ANALYTE DETECTION

(71) Applicant: INDIAN INSTITUTE OF TECHNOLOGY MADRAS (IIT MADRAS), Chennai (IN)

(72) Inventors: Raghavendra SAI, CHENNAI (IN); Sruthi PRASOOD USHA, Chennai (IN); HARIHARAN MANOHARAN, Chennai (IN)

(21) Appl. No.: 18/725,074

(22) PCT Filed: Jan. 2, 2023
(86) PCT No.: PCT/IN2023/050003

§ 371 Date: Jun. 27, 2024

(30) Foreign Application Priority Data
Dec. 31, 2021 (IN) 202141062168

(51) Int. Cl. G01N 21/552; G01N 21/77; G01N 33/543

(52) U.S. Cl. CPC G01N 21/554; G01N 21/7703; G01N 33/54346; G01N 33/54373; G01N 2021/7759

(57) ABSTRACT

The invention discloses a LSPR based label free immunosensing technique using a fiber optic system (100) for detecting an analyte molecule in a sample. The invention further discloses a U-shaped plasmonic optic fiber probe biosensor (200) to detect ochratoxin-A (OTA) in a sample and a method (300) of fabrication thereof. The optic fiber probe (101) biosensor (200) includes a sensing layer (205), a light source (102) to send light through the probe (101) and an optical detector (103) to detect a change in optical intensity due to a change in the localized surface plasmon resonance (LSPR) caused by binding of the analyte molecule to the antibody encapsulated in a metal organic framework. The antibody (204) in the sensing layer (205) is specific to the analyte molecule and configured to form an immunocomplex therewith.

### CROSS-REFERENCES TO RELATED APPLICATIONS

This application claims priority to provisional patent application No. 202141062168 entitled FIBER OPTIC BIOSENSOR FOR TRACE DETECTION OF OCHRATOXIN-A filed on 31 Dec. 2021.

### FIELD OF THE INVENTION

The present invention relates to fiber optic sensors and in particular to a system and method of fabricating optical fiber probes to sense analyte molecules of interest in a sample.

### DESCRIPTION OF RELATED ART

Ochratoxin-A (OTA) is a naturally occurring foodborne mycotoxin ubiquitously produced by fungi aspergillus and penicillium species. OTA is both chemically and physically stable molecule. It is generally present in daily food products such as fruits, nuts, spices, cereals, cocoa powder, processed juices, dairy products, beverages, infant foods, herbal products, and animal feeds under humid and hot storage conditions. Its presence in food causes more ill effects than a pesticide or a food additive, and ingestion leads to nephropathy and liver toxicity in humans and animals. Moreover, OTA has been classified as a group 2B potential human carcinogen by the International Agency for Research on Cancer 1993. Hence there is high need of on-the-spot quantifying techniques of OTA in food samples.

Conventional techniques used for quantification of OTA involve multiple intricate steps which make them expensive, sophisticated and more time consuming. However, on-site testing using labeled assay such as ELISA provides only semi-quantitative results. So there is need to develop portable label free sensor to detect OTA and similar mycotoxin species.

Metal-organic framework (MOF) is an emerging class of polymer, a hybrid material that contains metal ions coordinated to organic linkers to form a nanoporous structure. MOF-based biocomposites are suitable for sensing applications, as these molecules are capable of accommodating within their structure, both the analyte to be detected and the biorecognition unit (enzymes, antibodies, etc.) by infiltration within the porous network. Such structures could provide selectivity and sensitivity of detection of the target analyte, which is attractive for the fabrication of new diagnostic technologies such as point-of-care (POC) tests. It is reported that the zeolitic imidazole framework-8 (ZIF-8) acts as host matrix material for encapsulating biomolecules and ensures stable encapsulation of biorecognition units which can withstand a wide range of temperatures.

LiangLiang Liu et al. 2019 reported a tip-based fiber-optic localized surface plasmon resonance (LSPR) sensor anchored with metal organic framework (HKUST-1) film for sensing acetone. Bobin Lee et al. 2018, discusses an optical fiber based LSPR sensor coated with gold nanorods, for simple and rapid in-situ detection of Ochratoxin A. Hongmin Ma et al. 2016, discusses an immunosensor probe fabricated by doping silver nanoparticles over Pb (II) metal-organic framework encapsulating the antibody of Prostate-specific antigen (PSA) on the surface of MOF for detection of prostate cancer.

The current state of art provides plasmonic optical fiber sensor for OTA detection based on labeled competitive assay. However the label-free immune sensing of small molecule still remains a challenge, due to insufficient binding of molecule which produces meager change in the refractive index, hence lower the sensitivity and LOD of the sensor. There is therefore need for a LSPR based label-free immunosensor to detect small biomolecules such as OTA and other mycotoxins.

### SUMMARY OF THE INVENTION

Systems, devices and methods for detecting contaminant molecules in ultra-low trace quantities in a sample are disclosed. A fiber optic system (100) for detecting an analyte molecule in a sample is disclosed. The system (100) comprises a U-shaped plasmonic optic fiber probe (101) biosensor (200) configured to be immersed in a medium having an analyte of interest, wherein the U-shaped portion includes a sensing layer (205) comprising antibodies (204) specific to the analyte. The antibodies are encapsulated in a metal organic framework (203) deposited on gold nanoparticles, the sensing layer configured to detect a change in localized surface plasmon resonance (LSPR) property caused by binding of the analyte to the encapsulated antibodies. The system further includes a light source (102) connected to one leg of the U-bent probe (101), wherein the light source (102) is configured to send light through the probe (101) via a first optical fiber connector (104a), and an optical detector (103) connected to another leg of the U-bent probe through a second optical fiber connector (104b), wherein the optical detector (103) is configured to detect a change in optical intensity upon the interaction of the analyte with the sensing layer (205) of the biosensor (200), the change in intensity being proportional to the concentration of the analyte.

In some embodiments of the system, the fiber is a silica fiber or a polymer fiber. In some embodiments, the gold nanoparticles (201) are capped with a capping agent having carboxyl or hydroxyl groups, configured to allow crystallization of the metal organic framework (203) thereon. In some embodiments of the system, the metal organic framework (203) comprises a zeolitic imidazole framework (ZIF-8) including a zinc salt with imidazole as linker. In some embodiments of the system, the zeolitic imidazole framework (ZIF-8) (203) is configured to encapsulate the antibody (204). In some embodiments of the system, the functional groups on the gold nano particles (201) are configured to trap Zn²⁺ in ZIF-8 (203). In some embodiments of the system, the analyte is a small molecule of mass less than 1500 Da.

A U-shaped plasmonic optic fiber probe biosensor (200) configured to detect ochratoxin-A (OTA) in a sample, is disclosed in its various embodiments. The biosensor comprises a U-shaped optic fiber probe (101) having a sensing layer (205) coated with gold nanoparticles (201), wherein the gold nanoparticles (201) are configured to exhibit localized surface plasmon resonance (LSPR). An immobilised metal organic framework-antibody composite (202) is deposited on the coated optical fiber probe (101), wherein the composite (202) includes ochratoxin-A (OTA)-specific antibodies (204) encapsulated within the metal organic framework (203). The composite (202) is configured to capture OTA molecules, which produces a variation in LSPR property proportionate to the concentration of the OTA molecules in the sample, thereby altering transmission of light through the probe.

In some embodiments of the device, the fiber probe (101) is made of silica or polymer. In some embodiments of the device, the optic fiber probe (101) is coated with gold nanoparticles (201) of 30 nm size, immobilized over the probe (101) after amine functionalising. In some embodiments of the device, the metal organic framework (203) composite (202) comprises a zeolitic imidazole framework (ZIF-8) including a zinc salt with imidazole as linker, encapsulating OTA specific antibodies (204). In some embodiments of the device, the probe (101) is configured for label-free detection of OTA. In various embodiments, the detection of OTA molecule is carried out by observing the LSPR absorbance at peak absorbance wavelength in the range of the 545-585 nm. the In various embodiments, detecting range of OTA in the sample is 1 fg/ml-10 μg/ml. In some embodiments, the limit of detection (LOD) for OTA is 1 fg/ml or less.

A method of fabricating a fiber optic probe (300) configured to detect a small molecule analyte in a sample using localized surface plasmon resonance (LSPR) is disclosed. The method comprises the steps of providing (302) U-shaped optical fibre probe functionalized with terminal amine functional groups, immobilizing (303) citrate-capped gold nano particles (201) of 30 nm size over the functionalized optical probe surface, synthesizing (304) a metal organic framework-antibody composite by a one pot solvation method, comprising adding a analyte-specific antibody at a specified concentration to a solution containing zinc nitrate hexahydrate and 2-methylimidazole and depositing (306) in situ the metal organic framework-antibody composite on the probe surface, by exposure to the metal organic framework-antibody composite solution for a predetermined time, including encapsulating the antibody within the zeolitic imidazole framework on the gold nano particles, to obtain the fiber optic probe.

In various embodiments of the method, the analyte is a small molecule with a molecular mass of 1500 Da or less. In some embodiments, the step of immobilizing the gold nanoparticles (304) comprises exposing the amine-functionalized probe to an aqueous solution of gold nanoparticles capped with a capping agent having carboxyl or hydroxyl groups for 10-15 minutes at room temperature. In some embodiments, the depositing (306) comprises exposing the probe surface for a predetermined time of 1-2 hours and monitoring the capture of the antibody on the probe surface using LSPR until saturation in absorbance is reached. In various embodiments, the synthesizing (304) comprises adding the zinc salt (x), organic ligand (y) and antibody (z) in a molar ratio of x=1:y=4:z, where ‘z’ may vary in the range 1×10⁻³ to 20×10⁻³.

In some embodiments of the method, the analyte is ochratoxin-A (OTA) and the synthesizing (304) comprises adding the antibody at a concentration in the range 250-750 μg/ml.

This and other aspects are disclosed herein.

### BRIEF DESCRIPTION OF THE DRAWINGS

The invention has other advantages and features which will be more readily apparent from the following detailed description of the invention and the appended claims, when taken in conjunction with the accompanying drawings, in which:

FIG. 1A is a schematic representation of fiber optic system.

FIG. 1B enlarged view of U-bent Optical fiber sensor showing the sensing platform.

FIG. 2 illustrates a method for fabricating an optic fiber probe.

FIG. 3A shows the SEM image of AuNPs.

FIG. 3B shows the SEM image of U-bent optical fiber surface coated with in situ crystallization of ZIF-8 s over AuNP.

FIG. 3C shows the SEM image of U-bent optical fiber surface coated with in-situ crystallization of Ab@ZIF-8 over AuNP.

FIG. 4A shows LSPR peak wavelength for the probe having composite sensing layer with varying Ab concentration.

FIG. 4B shows saturated response for facile coverage of Ab@ZIF-8 over AuNP layer on the U-bent regime.

FIG. 4C shows LSPR absorbance values with varying OTA concentration.

FIG. 5A shows sensor response through different stages of probe fabrication.

FIG. 5B illustrates specificity analysis of the developed sensor probe by exposing to ZEA spiked in GPBS.

FIG. 5C shows selectivity analysis of developed probe by exposing to OTA+ZEA spiked in GPBS

FIG. 5D represents the repeatability analysis of AuNPs/Ab_{500 μg/ml}@ZIF-8 probe for random concentrations of OTA spiked in GPBS.

FIG. 6A shows the LSPR absorbance spectra for OTA concentrations from 1 fg/ml to 10 μg/ml.

FIG. 6B shows dose response curve of the developed sensor for OTA samples prepared in PBS buffer.

FIG. 6C shows linear response range which evaluated the sensitivity as 0.04246 at a peak absorbance wavelength of 575 nm.

FIG. 7A shows sensor response of the U-bent LSPR probe for OTA concentrations spiked in GPBS.

FIG. 7B shows comparison of the sensor response observed for OTA samples prepared in PBS and GPBS.

### DETAILED DESCRIPTION OF THE EMBODIMENTS

While the invention has been disclosed with reference to certain embodiments, it will be understood by those skilled in the art that various changes may be made and equivalents may be substituted without departing from the scope of the invention. In addition, many modifications may be made to adapt to a particular situation or material to the teachings of the invention without departing from its scope.

Throughout the specification and claims, the following terms take the meanings explicitly associated herein unless the context clearly dictates otherwise. The meaning of “a”, “an”, and “the” include plural references. The meaning of “in” includes “in” and “on.” Referring to the drawings, like numbers indicate like parts throughout the views. Additionally, a reference to the singular includes a reference to the plural unless otherwise stated or inconsistent with the disclosure herein.

Definitions: Throughout this specification, “small molecule” means any molecule having a molecular mass less than about 1500 Da. “Analyte” means any chemical or biological molecule that is capable of generating an antibody response in a human or animal. The toxin may be a mycotoxin found in food material for human or animal consumption.

The invention in its various embodiments discloses a fiber optic system for detecting an analyte in a sample. In various embodiments, the analyte may be a small molecule of molecular mass up to 1500 Daltons (Da). In some embodiments, the analyte may be a toxin such as a mycotoxin. In various embodiments, the invention further discloses a U-shaped plasmonic optic fiber probe biosensor and method of fabricating the probe configured for label free detection of the analyte in a sample. In various embodiments, the analyte may be ochratoxin-A, and the sample may be a food sample intended for human or animal consumption. The optic fiber probe may be made of silica or polymer. The probe region of the U-shaped plasmonic optic fiber probe may be coated with a sensing layer comprising an antibody immobilized within a metal organic framework-composite deposited over the optic fiber probe. In various embodiments, the antibody may be specific to the analyte to be detected. In one embodiment, the antibody may be specific to ochratoxin-A, and the sensor may be configured to detect ochratoxin-A in a food sample. A method of fabricating the sensor is further disclosed.

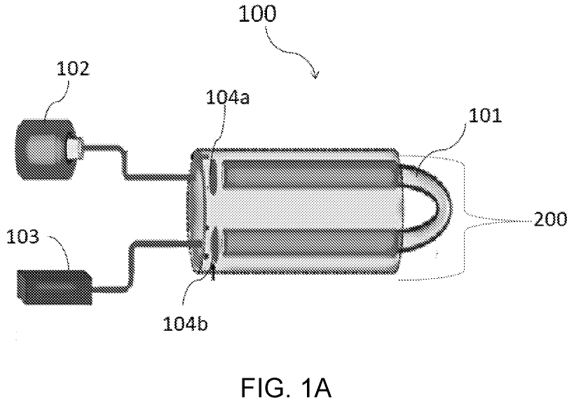

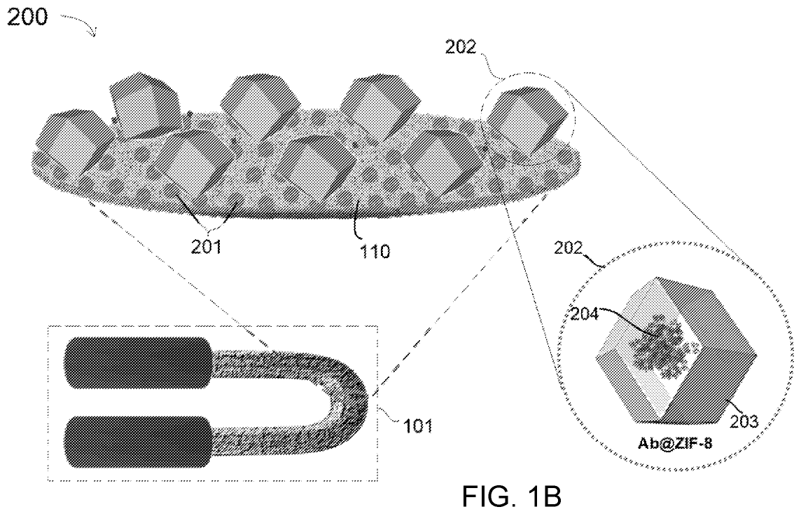

In various embodiments, the systems, devices and methods for detecting an analyte in a sample are further disclosed with reference to the figures. The sample may be any carrying medium having the analyte of interest. In various embodiments, the fiber optic system 100 is discussed with reference to FIGS. 1A and 1B. In various embodiments, the U-shaped plasmonic optic fiber probe biosensor 200 as shown in FIG. 1A includes U-shaped probe body portion 101 having a surface 110 with a sensing layer 205, a light source 102 connected to one leg of the U-bent probe 101 via a first optical fiber connector 104a and an optical detector 103 connected to the other leg of the U-bent probe through a second optical fiber connector 104b. The U-shaped plasmonic optic fiber probe 101 biosensor 200 is configured to be immersed in a medium having the analyte of interest. The immersion of the biosensor in the medium may be configured to bind the analyte to the antibody 204 immobilized in the composite 202. The light source 102 is configured to send light through the probe 101 via the first optical fiber connector 104a and the optical detector 103 is configured to receive light from the probe via the second optical connector 104b after interaction with the sample. In various embodiments, the optical detector 103 is configured to detect a change in optical intensity after interaction with the sensing layer 205 of the biosensor 200 via localized surface plasmon resonance (LSPR). The system is configured to detect the change in intensity being proportional to the concentration of the analyte being detected.

In various embodiments, the light source 102 is configured to be a laser, an LED source, or any other monochromatic light source. In various embodiments, the optical detector 103 may be a spectrometer, a CCD camera, or other suitable optical sensor configured to detect a change in intensity when compared with a standard sample.

In some embodiments the sensing layer 205 comprises a composite 202 encapsulating an antibody 204 in a metal organic framework (MOF) 203 deposited over the optic fiber probe 101, wherein the antibody 204 is specific to the analyte or antigen.

In some embodiments the optical fiber is a silica fiber and the optic fiber probe 101 coated with gold nanoparticles 201 is configured to allow crystallization of the metal organic framework 203 thereon. In some embodiments, the AuNPs may be of 30 nm diameter. In some embodiments, the AuNPs 201 may be capped with a capping agent having carboxyl or hydroxyl functional groups. In some embodiments the capping agent may be a derivative of citric acid or tannic acid. The gold nanoparticles 201 may be further configured to exhibit localized surface plasmon resonance on light passing through the optical fiber probe 101. In some embodiments, the metal organic framework 203 in the composite 202 comprises a zeolitic imidazole framework (ZIF-8) including a zinc salt with imidazole as linker. The composite 202 is configured to form a self-assembled monolayer with regulated pore structure such that the zeolitic imidazole framework (ZIF-8) 203 with the encapsulated antibody captures the analyte. In various embodiments, the functional groups in the capping agent may be configured to trap Zn²⁺ in ZIF-8 203 and to facilitate nucleation and growth of ZIF-8 203 over the probe surface.

In some embodiments, the plasmonic optical fiber probe sensor 200 may be configured for label-free detection of a mycotoxin, ochratoxin-A (OTA) in a sample. In some embodiments, the sensing layer 205 of the probe 101 comprises an immobilized OTA specific composite 202 in a ZIF-8 matrix 203 deposited over the optic fiber probe 101. In some embodiments, the probe 101 is configured to capture OTA molecules and produce a variation in LSPR proportionate to the concentration of the OTA molecules in the sample, on transmission of light through the probe.

In some embodiments, the fiber probe 101 is a silica fiber or polymer fiber. In various embodiments, the optic fiber probe 101 is coated with gold nanoparticles 201 immobilized over the probe 101. In some embodiments, the nanoparticles 201 may be capped with a capping agent having carboxyl or hydroxyl functional groups. In some embodiments, the functional groups on the gold nano particles 201 are configured to trap Zn²⁺ in ZIF-8 (203). In some embodiments the capping agent may be a derivative of citric acid or tannic acid. In some embodiments, the AuNPs may be immobilized over the probe after amine functionalizing. In some embodiments, the carboxylic acid group in the citrate-capped gold nano particles 201 are configured to trap Zn²⁺ in ZIF-8 203 and to facilitate nucleation and growth of ZIF-8 203 over the probe surface 110. In some embodiments, the AuNPs may be of 30 nm diameter. In some embodiments, the ZIF-8 framework 203 is configured to ensure tight encapsulation of antibodies 204 over the probe (101) surface.

In various embodiments, the invention discloses a method of detecting OTA species using the sensor probes 200 in a label-free format. The method involves dipping the probe 200 for specified time in a solution containing unknown concentration of the OTA species and monitoring the LSPR absorbance at peak absorbance wavelength in the range 545-585 nm. The method may include estimating the concentration of the OTA in the sample by observing the magnitude of the absorbance. In some embodiments, the method may comprise exposing the sensor probe 200 to a sample containing OTA until saturation of LSPR absorbance is achieved. In some embodiments, the saturation may be achieved in 20 minutes or less. In various embodiments of the method, the detecting range of OTA in a sample is 1 fg/ml-10 μg/ml. In various embodiments of the method, the lower limit of detection (LOD) for OTA in a sample is 1 fg/ml or less.

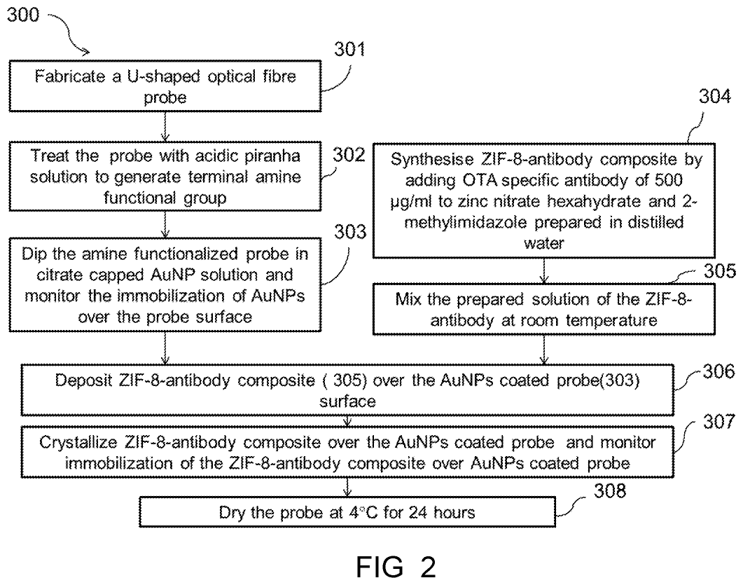

In various embodiments a method of fabricating the plasmonic optic fiber probe 101 of FIGS. 1A and 1B is disclosed with reference to FIG. 2. The method 300 includes in step 301 providing a U-shaped optical fiber to form the sensor region. The U-shaped optical fiber used in step 301 may in various embodiments be a silica optical fiber. In step 302 terminal amine functionalization is provided on the U-shaped optic fiber probe. The terminal amine functionalization in step 302 is done by treating the probe with acidic piranha solution, followed by APTMES solution. In step 303, citrate capped Au (gold) nanoparticles are coated on the optical fiber probe. In various embodiments, step 303 includes dipping the ammine functionalized probe in citrate capped AuNPs solution using chemisorption, for 10-15 minutes at room temperature. During step 303, the immobilization of the AuNPs on the fiber optic is monitored by passing light through the fibers and observing plasmonic absorbance peak shifts by a spectrometer, until saturation of absorbance is reached.

In various embodiments the synthesis of the metal ion framework-antibody composite through one pot solvation method in step 304, includes addition of the analyte-specific antibody to zinc nitrate hexahydrate and 2-methylimidazole solution prepared in distilled water, followed by self-assembly of the ZIF-8 molecules over the antibody particles to form a tight encapsulation. In various embodiments of the method, the zinc salt, organic framework and antibody may be zinc salt (x), organic ligand (y) and antibody (z) in a molar ratio of x=1:y=4:z, where ‘z’ may vary in the range 1×10⁻³ to 20×10⁻³. In various embodiments, the metal ion framework-antibody composite is mixed in step 305 is immediately deposited over the AuNPs coated probe surface. During step 307, the immobilization of the ZIF-8-antibody composite on Au nanoparticle coated fiber optic is monitored by passing light through the fibers and observing plasmonic absorbance peak shifts by a spectrometer. In one embodiment the immobilization of the ZIF-8-antibody composite over AuNP coated probe is done for 1-2 hours or until saturation of absorbance is reached.

In some embodiments of the method, the sensor may be configured to detect ochratoxin-A and the amount of OTA-specific antibody in step 304 may be in the concentration range 250-750 μg/ml. In one embodiment, the concentration of OTA-specific antibody in step 304 is 500 μg/ml.

In various embodiments of the method (300), the immobilization of ZIF-8-antibody composite over the AuNP coated probe surface includes in-situ crystallization of the ZIF-8-antibody composite.

In various embodiments of the method (300), optimum concentration of the antibody immobilized in the composite is identified in step 304 through the observation of saturation in evanescent wave absorption for plasmon peak in the range 400 to 700 nm.

In various embodiments of the method (300), the prepared probe may be dried at 4° C. for 24 hours after step 304, before use.

The invention has several advantages as set forth herein. The sensor system and sensor device of the invention has many advantages over the existing art. The technique presents an extremely sensitive method of detecting very small quantities of analytes, including mycotoxins, wherein the antibodies may be encapsulated in the metal organic framework. The analyte may enter the pores of the framework and may be detected by the antibody in a highly sensitive manner. The proposed fiber optic sensor establishes a label free immunoassay LSPR technique to detect ultralow or trace quantities of analyte molecules such as mycotoxins, including ochratoxin-A, and to quantify the same in a test sample. Although the examples illustrate detection of ochratoxin-A through immobilization of OTA-specific antibody, any other mycotoxin-specific or analyte-specific antibody may also be used to detect other mycotoxins or analyte. Here, the technique presents high specificity, as only the antibodies are involved in detection, metal oxide framework acts as an encapsulating agent only. Sensing may be achieved due to the binding of the analyte molecules with antibodies specific to the analyte being detected.

The capture of antibodies by the metal organic framework or matrix increases the loading efficiencies of antibodies over the gold nanoparticles. As mycotoxins are small molecules, the movement of them through the pores in the MOF is feasible, while it is not the same for bigger molecules of analytes, thus making the technique particularly suitable for detecting small molecules.

Moreover the sensing layer is stable due to electrostatic interaction between the antibodies and ZIF-8, and due to the stability of AuNPs which act as traps for the Zn²⁺ ions of the ZIF framework. The proposed system has helped to overcome the general limitations in direct label-free immunosensors, including limited stability, poor shelf life, low sensitivity and poor availability of antibodies on the probe surface for antigen binding.

### EXAMPLES

### Example 1: Fabrication of U-Bent Optical Fiber Probe

For plasmonic probe construction, optical fibers (FT400UMT, ThorLabs) of 400 μm core diameter and 0.39 NA procured from Thorlabs were used as the substrate to prepare U-bent probes. To construct the plasmonic probe, straight silica fibers of length 25 cm were taken and the buffer layer in the middle was stripped of using a sharp stainless steel razor. Then the fiber was then introduced to a butane flame and manual bending of the straight optical fiber was carried out to fabricate the U-bent fiber probe. Subsequently, the bent regime was dipped in acetone solution for 5 min to remove the polymer cladding and washed with excess water. Prior to any material deposition over the U-region, the probe had been cleaned with lint-free optical fiber wipes. The prepared U-bent probe has ˜1.4±0.2 mm bent diameter and it is 1 cm long. The constructed U-region in probe, going to act as the sensing region.

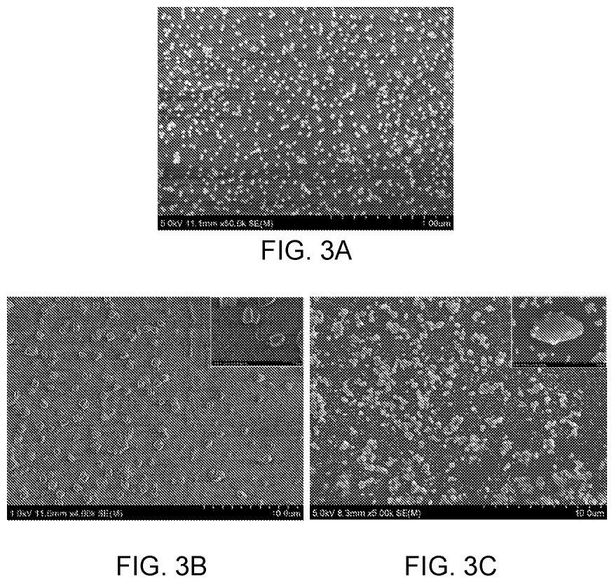

The next step in the fabrication of sensor probe includes immobilization of AuNPs over amine functionalized unclad core of U-bent optical fiber. To begin with, the probe surface was treated with acidic piranha solution (70% H₂SO₄: 30% H₂O₂; caution: piranha solution is highly corrosive) for 45 min at room temperature. This step generates silanol groups on the probe surface that covalently binds with the aminosilane molecules. Subsequently the probe was washed with excess DI water and kept in a hot air oven at 100° C. for 20 min so that moisture has been removed. The probe was dipped in APTMES (1% v/v, in 5:2 (v/v) of ethanol:acetic acid solvent) solution for 20 minutes, consequently the U-bent regime was washed and sonicated in ethanol, followed by drying in hot air oven for 45 min at 100° C. in order to remove unbound aminosilane molecules. Then the amine-functionalized U-bent silica probe was dipped in an aqueous AuNPs solution taken in glass vial which containing AuNPs of particle size 30 nm. The SEM micrograph of AuNPs of 30 nm size immobilized on the amine-functionalized probe surface is shown in FIG. 3A. The absorbance spectrum was recorded using Spectrasuite™ software. The real-time binding of AuNPs on amine functionalized surface of U-bent probe surface was monitored, which showed binding of the AuNPs to the probe surface was complete in 10-12 minutes (˜700 seconds).

The final step in probe fabrication was coating of sensing layer over the AuNP immobilized probe surface. For the preparation of composite, the one-pot solvothermal method was chosen for the in-situ synthesis of Ab@ZIF-8. Briefly, 10 μl of chosen Ab concentration was added to 70 μl of 40 mM zinc nitrate hexahydrate and 70 μl of 160 mM 2-methylimidazole prepared in distilled water. The solution was mixed at room temperature and immediately introduced to the AuNP immobilized probe surface. SEM micrographs of ZIF-8 over AuNP and Ab@ZIF-8 over AuNP immobilized over optical fiber surface are shown in FIGS. 3B and 3C respectively. The binding of the Ab@ZIF-8 was carried out under online monitoring until immobilization occur, duration for this was 1.5 hours. The prepared probe was dried at 4° C. for 24 h before experimentation.

### Example 2: Characterization Studies Performed for Fabrication of U-Bent Plasmonic Probe

### A) Evaluation of Surface Characteristics of Probes

Surface morphology of U-bent optical fiber surface coated with the AuNPs of 30 nm size immobilized on the amine-functionalized probe surface, in situ crystallization of ZIF-8 s over AuNP and Ab@ZIF-8 over AuNP were characterized using scanning electron microscope (SEM) imaging are shown in FIGS. 3(a) to 3(c), respectively.

### B) Evaluation of Binding of AuNPs and ZIF-8 Composite Over Probe Surface

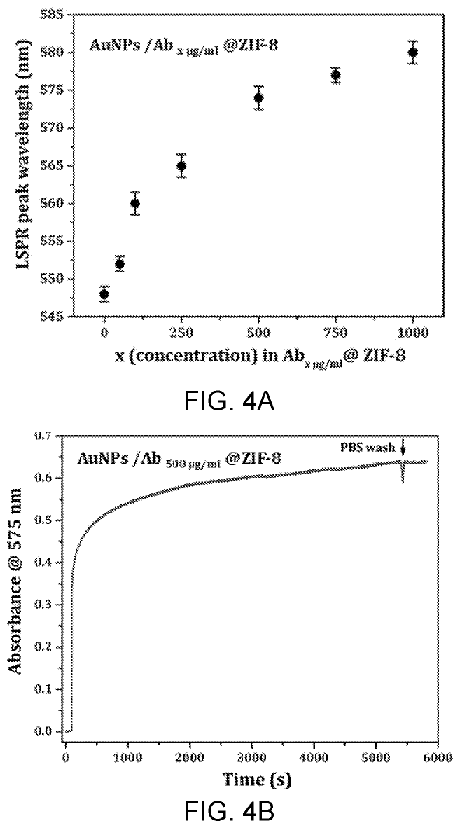

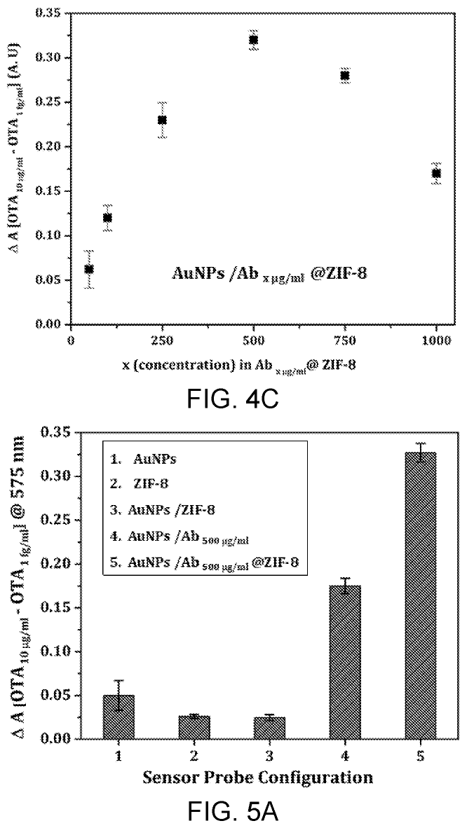

The real-time binding of AuNPs on amine functionalized surface of U-bent probe surface was monitored and plotted. The coating of ZIF-8 was carried out in real-time with online monitoring to evaluate the response from the probe. The binding of ZIF-8 resulted in a red shift in peak absorbance wavelength to 575 nm. Hence absorbance or sensor response was evaluated at 575 nm for the ZIF-8 incubation period. An immobilization period of 1.5 h was found to be sufficient to provide a stable and facile coverage of the sensing layer over the AuNPs layer on the U-bent regime as absorbance signal was saturated FIG. 4B. The absorbance spectrum and temporal response obtained for the optimized immobilization period of ZIF-8 are shown in FIGS. 4A and 4B respectively.

### C) Evaluation of Stability of Fabricated Probe

The stability of ZIF-8 in PBS was checked as OTA solutions were prepared in PBS. The absorbance spectrum was recorded after 10 sec and repeated after 1 hour of exposure of probe with AuNP/ZIF-8 in PBS. The responses were nearly identical, confirming the probe stability in PBS.

### D) Evaluation of Optimum Ab Concentration in Probe

The concentration of Ab in the Ab@ZIF-8 composite layer was optimized to incorporate the possible number of antibodies on the probe surface for maximal binding capability with antigen (OTA) so that larger range of OTA concentrations chosen for the analysis. For this analysis, probes with varying Ab concentration from 0 to 1000 μg/ml were prepared. Sensor probes with different Ab@ZIF-8 over AuNPs were fabricated by the same procedure discussed earlier. The shift in LSPR peaks during the immobilization of the sensing layer was monitored online and the peak absorbance wavelength obtained for each probe with different concentrations of Ab in ZIF-8 is plotted in FIG. 4A. The change in sensor response for the change in OTA concentration from 1 fg/ml to 10 μg/ml was evaluated for the sensor probe configuration with different Ab@ZIF-8 concentrations and results are plotted as shown in FIG. 4C. This confirmed the binding of OTA with Ab. A consistent red shift in the peak absorbance wavelength was observed with the increase in Ab concentration during the immobilization of Ab@ZIF-8 over AuNPs as well as with OTA binding. This is due to the increase in the effective RI of the medium surrounding AuNPs with the addition of Ab@ZIF-8 and OTA. The error bars represent the standard deviation from three iterations. A maximum absorbance change has been observed for the Ab concentration of 500 μg/ml in ZIF-8, which is due to the maximal availability and minimal aggregation of the Ab in the recognition medium to provide efficient binding of the analyte. Hence 500 μg/ml of Ab has been optimized for final probe fabrication. However, significant absorbance change was observed in the range 250-750 μg/ml, as evident in FIG. 4C.

### E) Evaluation of Sensing Parameters of Probe:

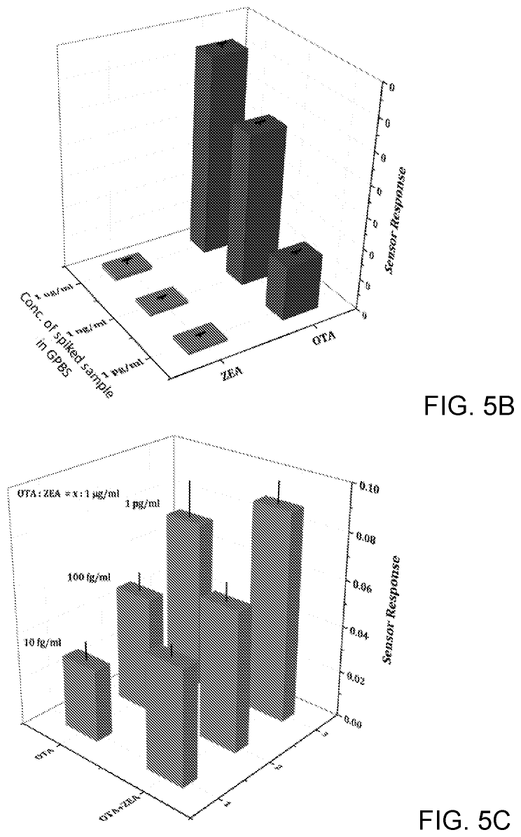

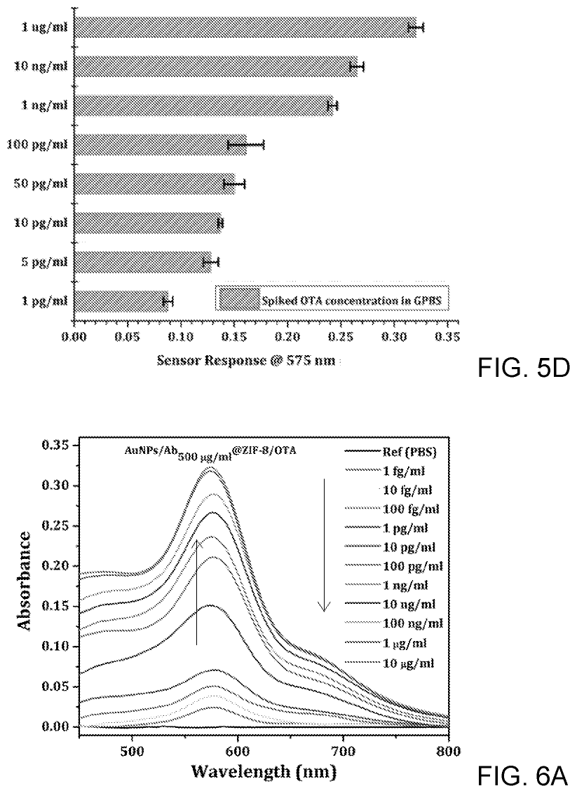

1) Test for performance: As control experiment, the sensing performances of the probes with distinct combinations of AuNPs, ZIF-8, AuNPs/ZIF-8, AuNPs/Ab (500 μg/ml) and AuNPs/Ab (500 μg/ml) @ZIF-8 were explored. The responses were analyzed in terms of variations in the spectral absorbance at 575 nm wavelength for the OTA concentration, from 1 fg/ml and 10 μg/ml, and plotted, as shown in FIG. 5A. A negligible shift has been observed in the case of probes with only AuNPs and ZIF-8 over AuNPs, which is due to the effective change in the RI of the surrounding medium in the presence of OTA. Thus, the analysis showed that AuNP/Ab@ZIF-8 is the best configuration to provide efficient sensing of OTA in a label-free manner and also confirm that ZIF-8 do not have an affinity for OTA molecules.

Based on the results from the experimental results obtained using a spectrometer as the detector in this study, similar to our previous study (Ramakrishna et al 2015, https://doi.org/10.1016/j.snb.2015.11.107), a LED-photodetector based configuration is anticipated to improve the LoDs much below 1 fg/mL as shown in our studies on attomolar analyte detection (Ramakrishna et al, 2020, https://doi.org/10.1016/j.snb.2020.128463).

2) Test for specificity: The fabricated probe was analysed for its specificity and selectivity towards OTA species. Zearalenone (ZEA), (MW: 318.36 g/mol) another mycotoxin of MW: 318.36 g/mol that coexists with OTA in many foods was chosen for the study. For this evaluation, samples were prepared by spiking specific concentrations of ZEA in GPBS with and without the presence of OTA. Three different concentrations of ZEA, 1 μg/ml, 1 ng/ml and 1 μg/ml were prepared and experimented using the developed sensor for specificity analysis. The LSPR absorbance responses observed are plotted and compared with the response observed for the respective concentrations of OTA as shown in FIG. 5B. The sensor is found to be highly specific to OTA.

3) Test for selectivity: For analyzing selectivity, GPBS samples were spiked with both OTA and ZEA. A higher ZEA concentration of 1 μg/ml was chosen and mixed with different OTA concentrations of 10 fg/ml, 100 fg/ml and 1 μg/ml. The sensor response obtained for the three samples, a comparative sensor response were made for sample of GPBS containing both OTA and ZEA with sample of GPBS spiked with OTA alone, as shown in are shown in FIG. 5C. The sensor can detect ultra-low OTA traces from food in the presence of other mycotoxins. Thus, the results are found to support the selective recognition of OTA in real samples.

4) Test for repeatability: The sensor showed good repeatability for OTA analysis in GPBS. FIG. 5D shows the bar diagram confirming the repeatability in the sensor responses for OTA analysis in GPBS. All the measurements were done in triplicate.

### Example 3: Experimental Setup on AuNPs/Ab@Zif-8 Layers Coated U-Bent Plasmonic Probe

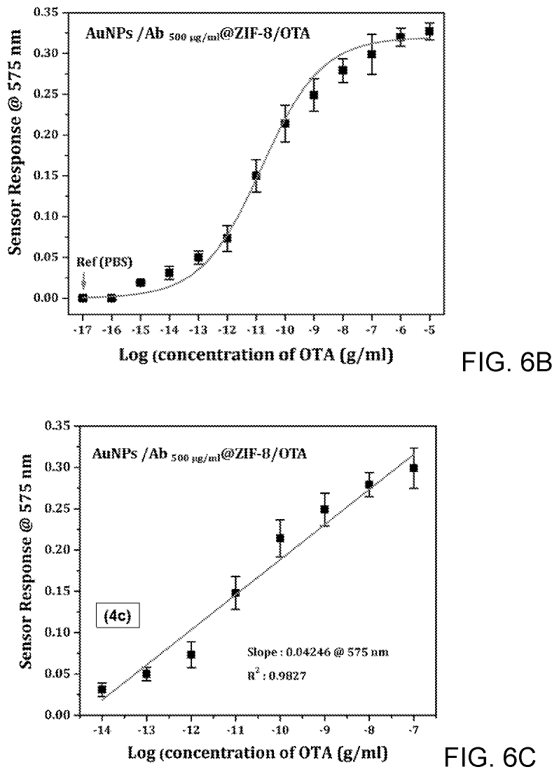

After fabrication & characterization of probe, one end of the U-bent probe was connected to tungsten halogen light source (HL-2000 from Ocean optics) using sub-miniature version-A (SMA905) connectors and bare fiber adapters and the other end was connected to CCD-array based fiber optic spectrometer (from 200 to 1100 nm wavelength, USB4000—XR1-ES, Ocean Optics Inc. USA). The entire device set up is shown in FIG. 1A. The U-bent fiber probe tip is then dipped into a custom-made glass sample holder/vial and sample analysis carried out in real-time with online monitoring. The materials procured for the study include AuNPs of 30 nm with optical density (OD)=1 from BBI Solutions, UK. OTA (analyte-1291/1) and rabbit polyclonal antibody specific to OTA (ab35133) were procured from Tocris Biosciences and Abcam, respectively. The precursors for ZIF-8, zinc nitrate hexahydrate and 2-methylimidazole, were obtained from Sigma Aldrich. OTA was obtained from Tocris. Due to its chemical nature, it can be dissolved either in an acidic or alkaline medium. Hence OTA was initially dissolved in methanol and then serially diluted in PBS to prepare concentrations from 1 fg/ml to 10 μg/ml (2.5 fM to 2.5 μM). LSPR absorption spectra were recorded using SpectraSuite® software. LSPR spectra obtained for the interaction of the probe with OTA samples were recorded and plotted as shown in FIG. 6A. The spectral response was evaluated at a wavelength of 575 nm, which was the LSPR peak observed for the optimized probe configuration with AuNPs, Ab of 500 μg/ml in ZIF-8 incubated for 1.5 h over AuNPs bound U-bent probe.

### Example:4 Real Time Analysis of OTA Samples Prepared in the Concentration Range from 1 fg/ml to 10 μg/m

Real-time for OTA samples were prepared in the concentration range from 1 fg/ml to 10 μg/ml. The LSPR spectral absorbance at 575 nm showed a steady increment in its value with an increase in the OTA concentration in the sample, as depicted in FIG. 6A. The sensor probe gave a saturated response in 20 mins. The dose-response, FIG. 6B, confirmed the saturation response of the sensor at higher OTA concentrations and a linear range was obtained over the OTA range from 10 fg/ml to 0.1 μg/ml (FIG. 6C). The sensitivity(S) was calculated from the slope of the linear fit as 0.0425 [Absorbance_{575 nm}/log (fg/ml)]. Using the conventional method for estimating the limit of detection (LOD), 3σ/S, the value was found to be 1 fg/ml for OTA. σ is the standard deviation calculated from the response by carrying out experiments in triplicate.

### Example:5 Real Time Analysis of for OTA Spiked Grape Juice Samples

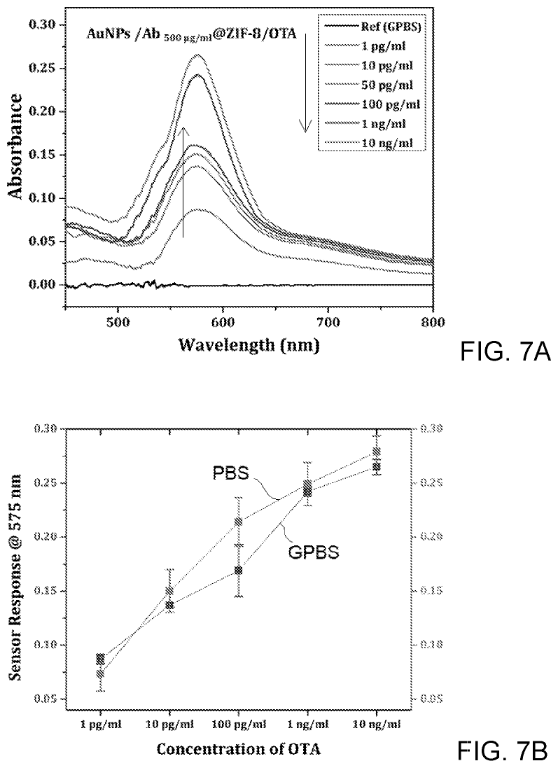

Real sample analysis was carried out using OTA concentrations spiked in grape juice sample bought from the market. The samples were prepared by spiking a specific concentration (1 pg/ml to 10 ng/ml) of OTA to a mixture of grape juice and PBS prepared in the ratio 1:1 (GPBS) and subjected to analysis. FIG. 7A shows the sensor response obtained for random concentrations of OTA spiked in GPBS. A comparison of the sensor response obtained by experimenting with similar concentrations of OTA spiked in GPBS and PBS is shown in FIG. 7B. The sensor response obtained for OTA samples spiked in GPBS shows that the results are in good agreement with that of OTA samples prepared in PBS. All the measurements were done in triplicate.

### Example: 6 Comparative Analysis of Current State of Art for OTA Sensors with Present Invention

The below table clearly emphasis that present invention is novel and establishes a label free immunoassay uses LSPR technique for OTA detection.

TABLE 1
Comparison of Labelled and Label-free OTA Assays using Plasmonic Transduction Techniques

| Sensor | | | Operational | | | Food | |
|---|---|---|---|---|---|---|---|
| configuration | Sensor Platform | Label | range | LOD | | sample | Reference |
| SPR/Direct | SPR chip/OTA | Label- | 0.1-20 | 0.028 | ng/ml | Dried fig | Akgönüllü et al., |
| assay | imprinted polymer | free | ng/ml | | | | 2022 |
| | film | | | | | | |
| SPR/ | SPR chip/OTA | Label- | 1.98-28.22 | 1.27 | ng/ml | Corn, | Wei et al (2019) |
| Competitive | antigen/OTA/Antibody | free | ng/ml | | | wheat | |
| assay | | | | | | | |
| LSPR/Direct | Optical fiber/ | Label- | 10 pM-100 | 12 | pM | Grape | Lee et al. (2018) |
| assay | Aunanorods/Aptamer | free | nM | | | juice | |
| LSPR/Direct | Solution | AuNPs | 0.0316-316 | 10^{-10.5} | g/ml | Corn | Liu et al. (2018) |
| assay | phase/AuNPs/ | | ng/ml | | | | |
| | Aptamer/NaCl | | | | | | |
| SPR/ | Prism/Gold | AuNPs | 0.1-1000 | 0.068 | ng/ml | Red | Karczmarczyk et |
| Competitive | film/OTA-bovine | | ng/ml | | | wine | al. (2016) |
| inhibition | serum albumin/ | | | | | | |
| assay | Antibody1/OTA/ | | | | | | |
| | Antibody2-AuNPs | | | | | | |
| SPR/ | SPR chip/OTA | Label- | 320-5700 | 160 | ng/mg | Barley | Joshi et al. (2016) |
| Competitive | antigen-Ovalbumin/ | free | ng/mg | | | | |
| assay | Antibody/OTA | | | | | | |
| LSPR/Direct | Glass slide/ | Label- | 1 nM-1 | ~1 | nM | Corn | Park et al. (2014) |
| assay | Aunanorods/Aptamer | free | μM | | | | (3) |
| LSPR/Direct | Optical fiber/AuNPs/ | Label- | 1 fg/ml-10 | 1.3 | fg/ml | Grape | Present |

| | | | | | |
|---|---|---|---|---|---|
| assay | Antibody@ZIF-8 | free | μg/ml | juice | invention |

We claim:

1. A fiber optic system (100) for detecting an analyte molecule in a sample, the system (100) comprising:
a U-shaped plasmonic optic fiber probe (101) biosensor (200) configured to be immersed in a medium having an analyte of interest, wherein the U-shaped portion includes a sensing layer (205) comprising antibodies (204) specific to the analyte encapsulated in a metal organic framework (203) deposited on gold nanoparticles, the sensing layer configured to detect a change in localized surface plasmon resonance (LSPR) property caused by binding of the analyte to the encapsulated antibodies;
a light source (102) connected to one leg of the U-bent probe (101), wherein the light source (102) is configured to send light through the probe (101) via a first optical fiber connector (104a); and
an optical detector (103) connected to another leg of the U-bent probe through a second optical fiber connector (104b), wherein the optical detector (103) is configured to detect a change in optical intensity upon the interaction of the analyte with the sensing layer (205) of the biosensor (200), the change in intensity being proportional to the concentration of the analyte.

2. The system (100) as claimed in claim 1, wherein the fiber is a silica or a polymer fiber.

3. The system (100) as claimed in claim 1, wherein the gold nanoparticles (201) are capped with a capping agent having carboxyl or hydroxyl groups, configured to allow crystallization of the metal organic framework (203) thereon.

4. The system (100) as claimed in claim 3, wherein the metal organic framework (203) comprises a zeolitic imidazole framework (ZIF-8) including a zinc salt with imidazole as linker.

5. The system (100) as claimed in claim 4, wherein the zeolitic imidazole framework (ZIF-8) (203) is configured to encapsulate the antibody (204).

6. The system (100) as claimed in claim 4, wherein the functional groups on the gold nano particles (201) are configured to trap Zn²⁺ in ZIF-8 (203)

7. The system (100) as claimed in claim 1, wherein the analyte is a small molecule of mass less than 1500 Da.

8. A U-shaped plasmonic optic fiber probe biosensor (200) configured to detect ochratoxin-A (OTA) in a sample, comprising:
a U-shaped optic fiber probe (101) having a sensing layer (205) coated with gold nanoparticles (201), wherein the gold nanoparticles (201) are configured to exhibit localized surface plasmon resonance (LSPR);
an immobilised metal organic framework-antibody composite (202) deposited on the coated optical fiber probe (101), wherein the composite (202) includes ochratoxin-A (OTA)-specific antibodies (204) encapsulated within a metal organic framework (203); wherein
the composite (202) is configured to capture OTA molecules and produce a variation in LSPR property proportionate to the concentration of the OTA molecules in the sample, thereby altering transmission of light through the probe.

9. The sensor (200) as claimed in claim 8, wherein the fiber probe (101) is made of silica or polymer.

10. The sensor (200) as claimed in claim 9, wherein the optic fiber probe (101) is coated with gold nanoparticles (201) of 30 nm size, immobilized over the probe (101) after amine functionalising.

11. The sensor (200) as claimed in claim 9, wherein the metal organic framework (203) composite (202) comprises a zeolitic imidazole framework (ZIF-8) including a zinc salt with imidazole as linker, encapsulating OTA specific antibodies (204).

12. The sensor (200) as claimed in claim 11, wherein the probe (101) is configured for label-free detection of OTA.

13. The sensor (200) as claimed in claim 8, wherein the detection of OTA molecule is carried out by observing the LSPR absorbance at peak absorbance wavelength in the range of the 545-585 nm.

14. The sensor (200) as claimed in claim 8, wherein the detecting range of OTA in the sample is 1 fg/ml-10 μg/ml.

15. The sensor (200) as claimed in claim 8, wherein the limit of detection (LOD) for OTA is 1 fg/ml or less.

16. A method of fabricating a fiber optic probe (300) configured to detect a small molecule analyte in a sample using localized surface plasmon resonance (LSPR) comprising:
providing (302) U-shaped optical fibre probe functionalized with terminal amine functional groups;
immobilizing (303) citrate-capped gold nano particles (201) of 30 nm size over the functionalized optical probe surface;
synthesizing (304) a metal organic framework-antibody composite by a one pot solvation method, comprising adding a analyte-specific antibody at a specified concentration to a solution containing zinc nitrate hexahydrate and 2-methylimidazole and
depositing (306) in situ the metal organic framework-antibody composite on the probe surface, by exposure to the metal organic framework-antibody composite solution for a predetermined time, including encapsulating the antibody within the zeolitic imidazole framework on the gold nano particles, to obtain the fiber optic probe.

17. The method (300) as claimed in claim 16, wherein the analyte is a small molecule with a molecular mass of 1500 Da or less.

18. The method (300) as claimed in claim 16, wherein immobilizing the gold nanoparticles (304) comprises exposing the amine-functionalized probe to an aqueous solution of gold nanoparticles capped with a capping agent having carboxyl or hydroxyl groups for 10-15 minutes at room temperature.

19. The method (300) as claimed in claim 16, wherein the depositing (306) comprises exposing the probe surface for a predetermined time of 1-2 hours and monitoring the capture of the antibody on the probe surface using LSPR until saturation in absorbance is reached.

20. The method (300) as claimed in claim 16, wherein the synthesizing (304) comprises adding the zinc salt (x), organic ligand (y) and antibody (z) in a molar ratio of x=1:y=4:z, where ‘z’ may vary in the range 1×10⁻³ to 20×10⁻³.

21. The method (300) as claimed in claim 16, wherein the analyte is ochratoxin-A (OTA) and the synthesizing (304) comprises adding the antibody at a concentration in the range 250-750 μg/ml.
